(19) United States
(12) Patent Application Publication
(10) Pub. No.: US 2017/0360501 A1
(43) Pub. Date: Dec. 21, 2017

(54) DISPOSABLE BIPOLAR COAXIAL RADIO FREQUENCY ABLATION NEEDLE, SYSTEM AND METHOD

(71) Applicant: Daniel Igor Branovan, Livingston, NJ (US)

(72) Inventor: Daniel Igor Branovan, Livingston, NJ (US)

(21) Appl. No.: 15/188,952

(22) Filed: Jun. 21, 2016

(51) Int. Cl. A61B 18/14; A61B 18/12; A61B 18/00

(52) U.S. Cl. CPC A61B 18/1477; A61B 18/1206; A61B 2018/00184; A61B 2018/1475; A61B 2018/143

(57) ABSTRACT

Some implementations include a coaxial bipolar ablation instrument comprising an outer needle and an inner needle inserted into the outer needle. The instrument can also include a first insulating layer disposed between the inner needle and the outer needle, and a second insulating layer disposed over a portion of the outer needle. The instrument can further include a first exposed region of the instrument disposed near a proximate end of the instrument, the first exposed region including a first exposed portion of the inner needle and a first exposed portion of the outer needle, the first exposed region constructed to provide contact to a connector, and a second exposed region of the instrument disposed near a distal end of the instrument, the second exposed region forming the active region of the instrument and including a second exposed portion of the inner needle and a second exposed portion of the outer needle.

### FIELD

Embodiments relate generally to electrical surgical instruments, and, more particularly, to disposable bipolar coaxial radio frequency ablation instruments (e.g., needles), systems and methods of using the same.

### BACKGROUND

A common treatment for malignant tumors in human organs, such as nodules on the thyroid or renal masses on the kidney, is surgical removal of most of the respective organ tissue. For example, a thyroidectomy may be performed to deal with malignant thyroid tumors, a procedure which unfortunately results in removal of most of the thyroid tissue. Moreover, undergoing thyroid surgery often poses risks, such as nerve damage or damage to parathyroid glands, and may require that the patient take thyroid hormone supplements following surgery. Alternatives to thyroidectomy are known in the art, including radio frequency (RF) ablation techniques in which the temperature of the target tissue may be raised to a temperature of 50° C. or higher.

For example, Holmer et al. have evaluated ablation methods, as reported in “Bipolar Radiofrequency Ablation for Nodular Thyroid Disease—ex Vivo and in Vivo Evaluation of a Dose-Response Relationship,” J. Surg. Res. 2009 Oct. 29. A study in percutaneous RF ablation for benign thyroid nodules was also described by Kim et al. in “Radiofrequency Ablation of Benign Cold Thyroid Nodules: Initial Clinical Experience,” Thyroid, 2006 April, 16(4):361-7. Kim et al. reported that the ablation electrode used was internally cooled, and that a majority of the patients required conscious sedation when undergoing the ablation procedure.

While there are known in the art RF devices suitable for use in the ablation of liver tumors, for example, most such devices require an extended period of use of from five to ten minutes per session. This length of time makes it impractical to use a conventional RF device on thyroid nodules, in particular, as the thyroid will move with the swallowing motions of the patient. A need may exist for an electrocautery or percutaneous ablation instrument that will allow for relatively quick excision of a malignant tissue or tumor.

The existing methods of treatment by high-frequency ablation (HFA) typically suggest the use of either mono-polar or bipolar method. In varying degrees, each of these techniques has certain limitations defined by the physical characteristics of the ablation instrument.

Thus with a mono-polar method, the patient's electrode must have a constant reliable electrical contact with the patient's body, which in some cases is not always easy to ensure or implementation or is not always convenient. Meanwhile, high-frequency currents flowing through the patient's body from the patient's electrode to an active electrode typically occur through extensive portions of healthy tissues and may have the additional undesirable effect on those tissues. Further, there could be a potential negative impact of these currents upon, for instance, a pacemaker.

Bipolar ablation requires existence of two electrodes located close to each other to have impact only on the portion of body located between these electrodes. Unlike monopolar ablation, the bipolar method allows to limit the impact of electric current only to the “area of interest”, and also substantially reduce the exposure time to achieve the desired result (coagulation size). In general, a tool for a bipolar ablation is used to work on the visible surface. Alternatively, special tools are used with thin parallel conductive electrodes (needles) creating a possibility of impact upon internal areas, such as patent BY 19188 C1 2015.06.30, used in the treatment of thyroid neoplasms. Nevertheless, the relative bulkiness of the tool, and the presence of two needles with the protruding edge of the insulator on each electrode hinders the penetration and control of their position in the exposure area, increases the size of the wound and may lead to undesirable emotions of the patient. In addition, the tool is difficult to sterilize, and may not be suitable as a disposable instrument (e.g., for single use).

### SUMMARY

Some implementations can include a coaxial bipolar ablation instrument comprising an outer needle and an inner needle inserted into the outer needle. The instrument can also include a first insulating layer disposed between the inner needle and the outer needle, and a second insulating layer disposed over a portion of the outer needle. The instrument can further include a first exposed region of the instrument disposed near a proximate end of the instrument, the first exposed region including a first exposed portion of the inner needle and a first exposed portion of the outer needle, the first exposed region constructed to provide contact to a connector, and a second exposed region of the instrument disposed near a distal end of the instrument, the second exposed region forming the active region of the instrument and including a second exposed portion of the inner needle and a second exposed portion of the outer needle.

The instrument can also include a connector coupled to the instrument and having a first contact contacting the first exposed portion of the outer needle and a second contact contacting the first exposed portion of the inner needle. The inner needle and the outer needle can be formed from stainless steel surgical needles.

The first insulating layer and the second insulating layer can be formed from medical lacquer. The second exposed region of the inner needle can be about 7 mm and the second exposed portion of the outer needle can be about 7 mm.

The first exposed portion can include an exposed portion of the first insulating layer. The second exposed portion can include an exposed portion of the first insulating layer. A diameter of the inner needle can be about 0.5 mm. A diameter of the outer needle can be about 0.9 mm.

Some implementations can include a high frequency ablation system having an electrosurgical generator, and a coaxial bipolar ablation instrument (as described above). The system can also include an adapter cable having a first end constructed to be coupled to the instrument and a second end constructed to connect to the electrosurgical generator, a power supply coupled to the electrosurgical generator; and a control switch coupled to the electrosurgical generator.

The system can also include a connector coupled to the instrument and having a first contact contacting the first exposed portion of the outer needle and a second contact contacting the first exposed portion of the inner needle. The inner needle and the outer needle can be formed from stainless steel surgical needles.

The first insulating layer and the second insulating layer can be formed from medical lacquer. The second exposed region of the inner needle can be about 7 mm and the second exposed portion of the outer needle can be about 7 mm.

The first exposed portion can include an exposed portion of the first insulating layer. The second exposed portion can include an exposed portion of the first insulating layer.

A diameter of the inner needle can be about 0.5 mm. A diameter of the outer needle can be about 0.9 mm.

In some implementations, an electrocautery instrument comprises: a handle having a handle axis; a first electrode assembly, the first electrode assembly having a first handle electrode section retained in the handle, a first oblique electrode section extending from the handle, and a first ablation electrode section disposed at an offset distance from the handle axis; and a second electrode assembly, the second electrode assembly having a second handle electrode section retained in the handle, a second oblique electrode section extending from the handle, and a second ablation electrode section disposed at the offset distance from the handle axis, the second electrode assembly being generally congruent to the first electrode assembly, the handle being configured to retain the first ablation electrode section in generally parallel relationship to the second ablation electrode section.

In some implementations, an electrocautery system comprises: a handle having a handle axis; a first electrode assembly partially retained in the handle, the first electrode assembly including a first active electrode distal from the handle; a second electrode assembly partially retained in the handle, the second electrode assembly including a second active electrode distal from the handle, the first active electrode retained in generally parallel relationship with the second active electrode so as to define a bipolar ablation zone therebetween, the bipolar ablation zone being in an offset and substantially parallel alignment with the handle axis; and an RF power supply electrically connected to the first electrode assembly and to the second electrode assembly, the RF power supply functioning to produce a predefined level of ablative RF power in the bipolar ablation zone.

Some implementations can include a method for ablating a tissue in a patient comprises the steps of: obtaining an instrument having both a first electrode assembly and a second electrode assembly retained in a handle, the handle retaining the first electrode section in a generally parallel relationship with the second electrode section so as to define a substantially linear bipolar ablation zone between a portion of the first electrode assembly and a portion of the second electrode assembly, the bipolar ablation zone being in an offset and substantially parallel alignment with an axis of the handle; inserting the bipolar ablation zone into a patient proximate a region containing the tissue; determining that a portion of the tissue has been positioned within the bipolar ablation zone; powering the first electrode assembly and the second electrode assembly for a predetermined time period so as to produce a predefined level of ablative RF power in the bipolar ablation zone; and removing the bipolar ablation zone from the patient.

Additional features and advantages of the disclosed subject matter are set forth in the detailed description which follows and/or will be apparent to those skilled in the art.

### BRIEF DESCRIPTION OF THE DRAWINGS

FIG. 1 is an isometric illustration of an electrocautery instrument comprising a handle and a pair of electrode assemblies, in accordance with some implementations;

FIG. 2 is a partial-cutaway of the electrocautery instrument of FIG. 1 showing blade contacts, handle electronic sections, and an electronic support insulator secured in the handle;

FIG. 3 is an enlarged view of an ablation electrode section in the electrocautery instrument of FIG. 1;

FIG. 4 is a flow diagram illustrating operation of the electrocautery instrument of FIG. 1;

FIG. 5 is a diagrammatical illustration of an ablating system utilizing the electrocautery instrument of FIG. 1;

FIG. 6 is a side view diagram of an exemplary embodiment of an electrocautery instrument, in accordance with the prior art;

FIG. 7 is a top view diagrammatical illustration of the electrocautery instrument of FIG. 6;

FIG. 8 is a diagrammatic illustration of an example ablating system utilizing a coaxial electrocautery instrument in accordance with some implementations;

FIG. 9 is a diagram of an example coaxial electrocautery instrument in accordance with some implementations;

FIG. 10 is a diagram of an example coaxial electrocautery instrument showing example dimensions in accordance with some implementations in accordance with some implementations; and

FIG. 11 is a flow diagram illustrating an example method of operation of the electrocautery instrument of FIG. 9 in accordance with some implementations.

FIG. 12 shows a diagram of an example coaxial bipolar ablation instrument and a simulated field pattern in accordance with some implementations.

FIG. 13 shows a diagram of an example coaxial bipolar ablation instrument having an integrated connector in accordance with some implementations.

### DETAILED DESCRIPTION

Some implementations can include a bipolar radio frequency (RF) ablation or electrocautery instrument designed for percutaneous ablation of tissue in a human cavity, such as thyroid nodules or renal masses. The instrument may be inserted through a patient's skin to thyroid nodules or to renal cell carcinomas under ultrasound guidance. Activation of the instrument serves to quickly destroy the malignant tissue. The bipolar configuration provides for the ability to localize the region of ablation and to thus minimize peripheral damage to surrounding, healthy tissue.

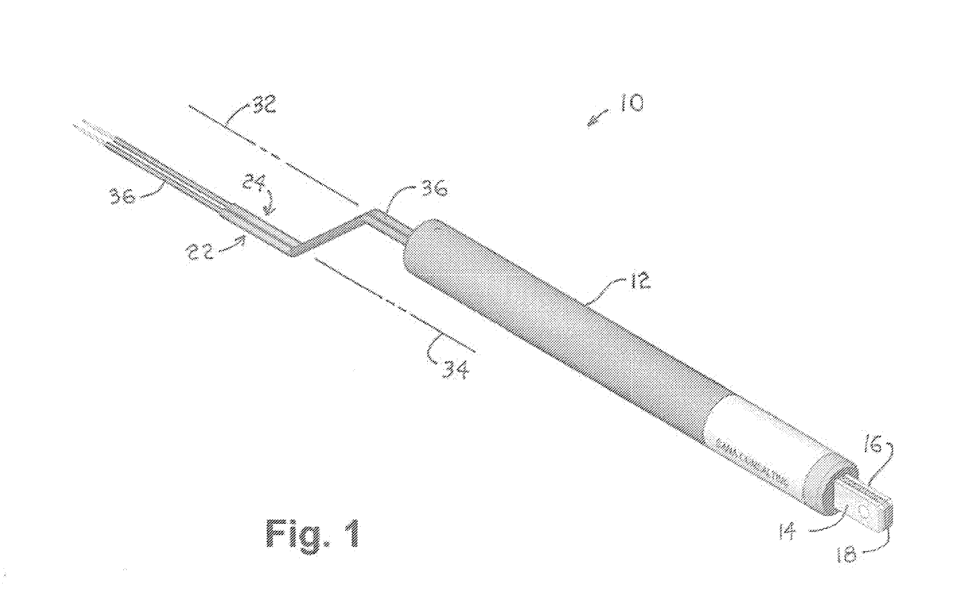

There is shown in FIG. 1 an exemplary embodiment of an electrocautery instrument 10 comprising a handle 12 retaining a first electrode assembly 22 and a second electrode assembly 24. The handle 12 may be fabricated from a nonconductive material, such as a plastic or dielectric. The first electrode assembly 22 and the second electrode assembly 24 are electrically connected to a first blade contact 14 and a second blade contact 16, respectively.

A blade insulation spacer 18 may be disposed between the first electrode assembly 22 and the second electrode assembly 24 so as to electrically isolate the first electrode assembly 22 from the second electrode assembly 24. The first electrode assembly 22 and the second electrode assembly 24 may thus be powered by applying RF power to the first blade contact 14 and the second blade contact 16.

As shown in the diagram, the portions of the first electrode assembly 22 and the second electrode assembly 24 distal from the handle 12 are in an offset configuration. These distal electrode assembly portions lie along an ablator axis 34 that is offset from a handle axis 32 that lies along a centerline of the handle 12. As can be appreciated by one skilled in the art, the offset configuration shown is particularly advantageous providing a clear view of the skin puncture site before insertion of the distal electrode assembly portions into the patient.

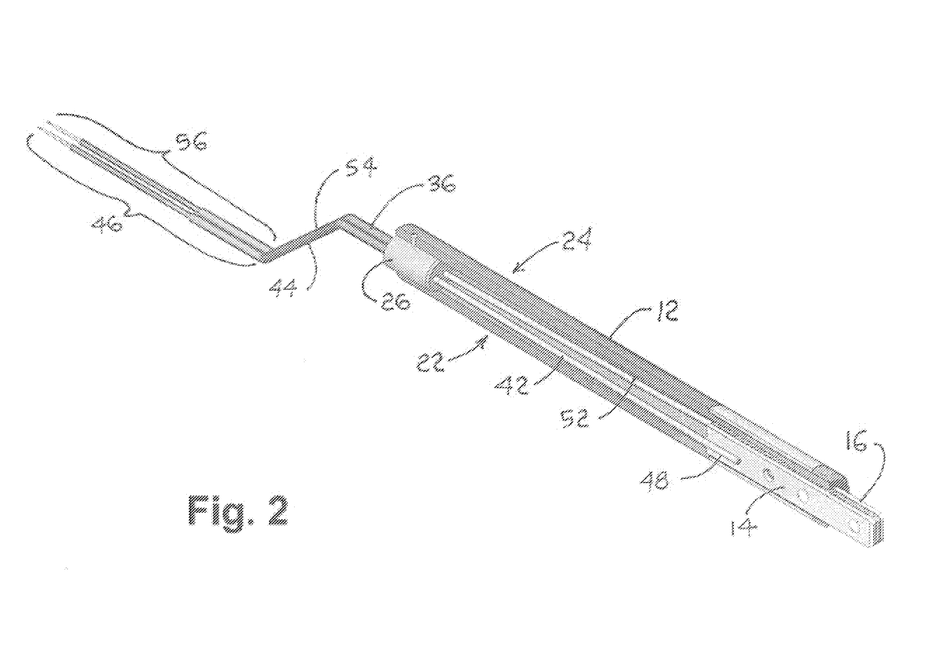

As shown in FIG. 2, the first electrode assembly 22 comprises a first handle electrode section 42, a first oblique electrode section 44, and a first ablation electrode section 46. The first handle electrode section 42 may be electrically connected to the first blade contact 14 at an electrical attachment 48, such as by brazing or welding. The second electrode assembly 24 is similar in configuration to the first electrode assembly 22. Accordingly, the second electrode assembly comprises a second handle electrode section 52, a second oblique electrode section 54, and a second ablation electrode section 56.

The handle 12 is configured to retain the first blade contact 14 and the second blade contact 16 at the rear of the handle 12. An electrode support insulator 26 may be provided at the front of the handle 12 to retain the first handle electrode section 42 and the second handle electrode section 52 in a spaced apart, substantially parallel relationship.

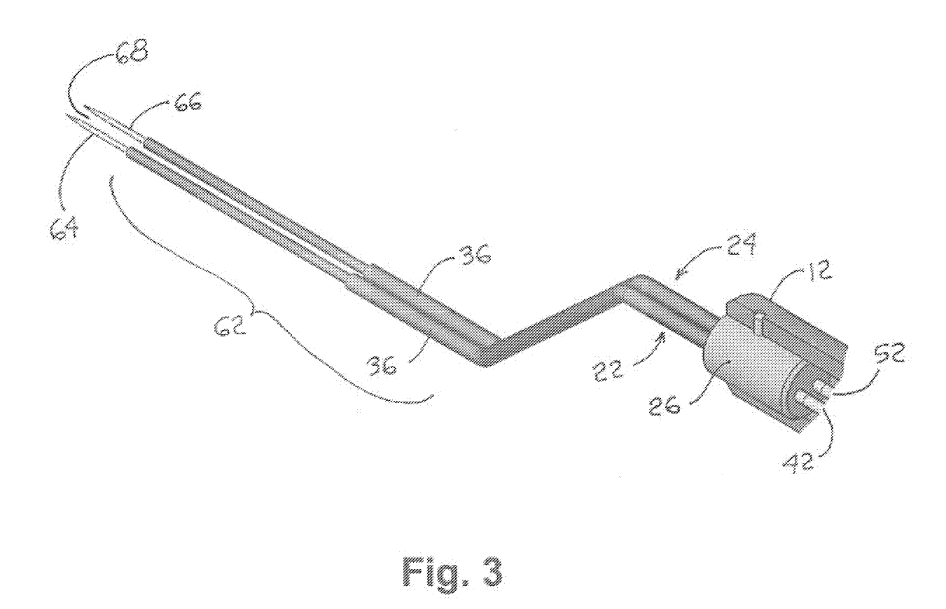

As best shown in FIG. 3, the first ablation electrode section 46 may be partially covered with the insulating sleeve 36 to form an insulated ablation electrode 62 for part of the length of the first ablation electrode section 46, and a first active electrode 64 without the insulating sleeve 36 for the remaining length of the first ablation electrode section 46. Similarly, the second ablation electrode section 56 may be partially covered with the insulating sleeve 36 to form a second active electrode 66, where a bipolar ablation zone 68 may be defined as the region between the first active electrode 64 and the second active electrode 66. This configuration serves to restrict any electrical discharge between the first electrode assembly 22 and the second electrode assembly 24 to the bipolar ablation zone 68.

It can be appreciated that the exposed lengths of the active electrodes 64, 66 determine the size of the resulting ablated lesion. The exposed lengths of the active electrodes 64, 66 are thus a function of the size of the target tumor. In an exemplary embodiment, the spacing between the first active electrode 64 and the second active electrode 66 is specified so as to be able to enclose a thyroid nodule or a renal carcinoma between the first active electrode 64 and the second active electrode 66 for cauterization by the electrocautery instrument 10.

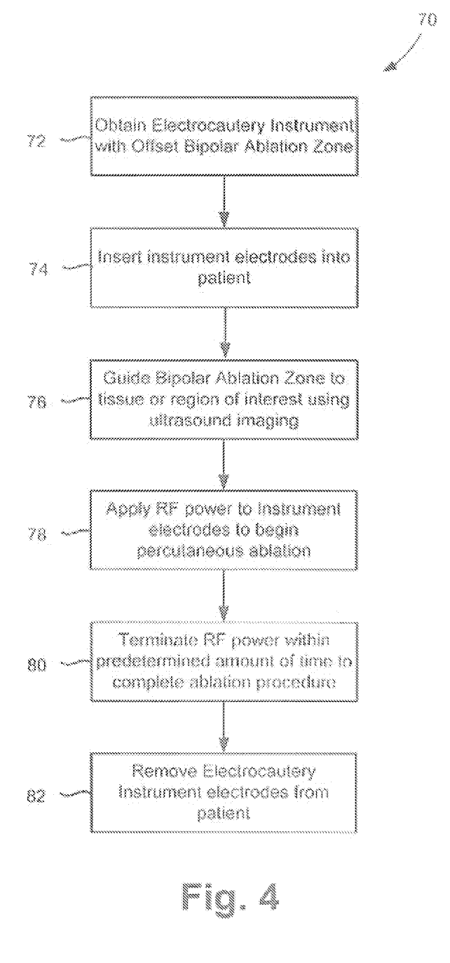

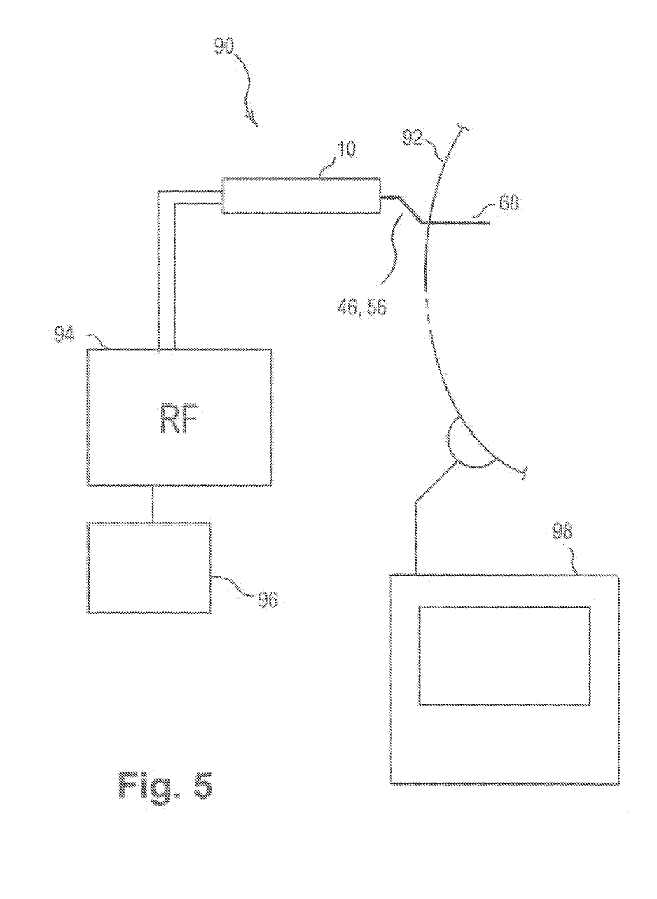

Operation of the electrocautery instrument 10 may be described with reference to a flow diagram 70, shown in FIG. 4, in which the electrocautery instrument 10 with the offset bipolar ablation zone 68 is obtained, at step 72. With additional reference to FIG. 5, the first ablation electrode section 46 and the second ablation electrode section 56 are inserted into a patient 92, at step 74. The bipolar ablation zone 68 may be guided to a target tissue or to a region of interest, such as a thyroid or a kidney, using feedback from an ultrasound imaging unit 98, at step 76.

It can be appreciated that, as the first ablation electrode section 46 and the second ablation electrode section 56 are formed from metal, the location of the first ablation electrode section 46 and the second ablation electrode section 56 inside the patient can be established by means of ultrasound imaging. Power may be applied to the electrocautery instrument 10, at step 78, using an RF power source 94 and control unit 96. In an exemplary embodiment, the RF power source 94 may output between about ten watts and twenty watts of RF power at an operating frequency of about 800 MHz to about 6.0 GHz.

The RF power source 94 may provide ablative energy to the bipolar ablation zone 68 for a predetermined period of time to complete the electrocautery or percutaneous ablation procedure, at step 80. In an exemplary embodiment, the predetermined period of time may comprise a duration of from about ten seconds to about thirty seconds. Because the electrocautery procedure can be completed within the upper time period of thirty seconds, it may not be necessary to have the patient placed under general anesthesia. The control unit 96 may be used to power down the RF power source 94 to terminate the ablation process. The first ablation electrode section 46 and the second ablation electrode section 56 may then be removed from the patient 92, at step 82.

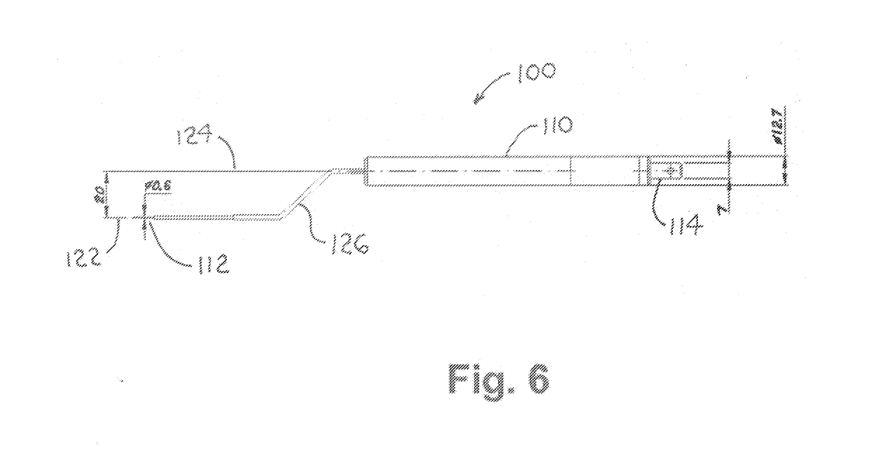

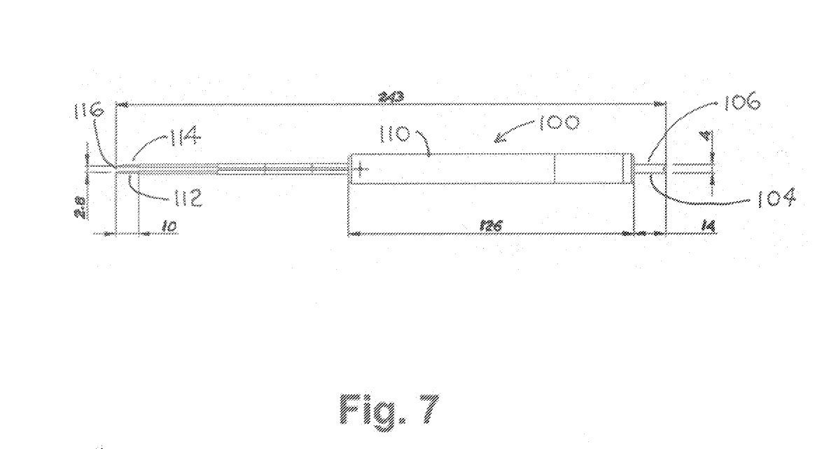

In an exemplary embodiment, an electrocautery instrument 100 may be fabricated as a device having an overall length of approximately 243 mm, as shown in FIGS. 6 and 7. The electrocautery instrument 100 may comprise a handle 110 approximately 126 mm in length and about 12.7 mm in diameter. A first blade contact 104 and a second blade contact 106 are configured to interface with standard RF power supplies and, accordingly, may each have a width of about 7.0 mm, protrude approximately 14 mm from the handle 110, and have outer surfaces spaced at a distance of about 4 mm.

The electrocautery instrument may comprise a first active electrode 112 and a second active electrode 114, each about 10 mm in length. The first active electrode 112 may be spaced from the second active electrode 114 by a distance of about 2.8 mm, although an alternative spacing of from about 2.2 mm to about 3.2 mm would lie within the scope of the present disclosure. This range of dimensions enables an optimal bipolar cautery to provide for a relatively quick ablation procedure. In addition, damage to surrounding tissue may be mitigated or eliminated by using the relatively quick procedure.

The diameters of the first active electrode 112 and the second active electrode 114 may be about 0.6 mm in diameter. The configuration shown provides for a bipolar ablation zone 116 of about 10 mm by about 2.2 mm. An ablator axis 122 may be offset from a handle axis 124 by a distance of about 20 mm as shown, although an alternative offset distance of from about 10 mm to about 30 mm would also lie within the scope of the present disclosure. An oblique electrode section 126 may form an angle of approximately 45° with the handle axis.

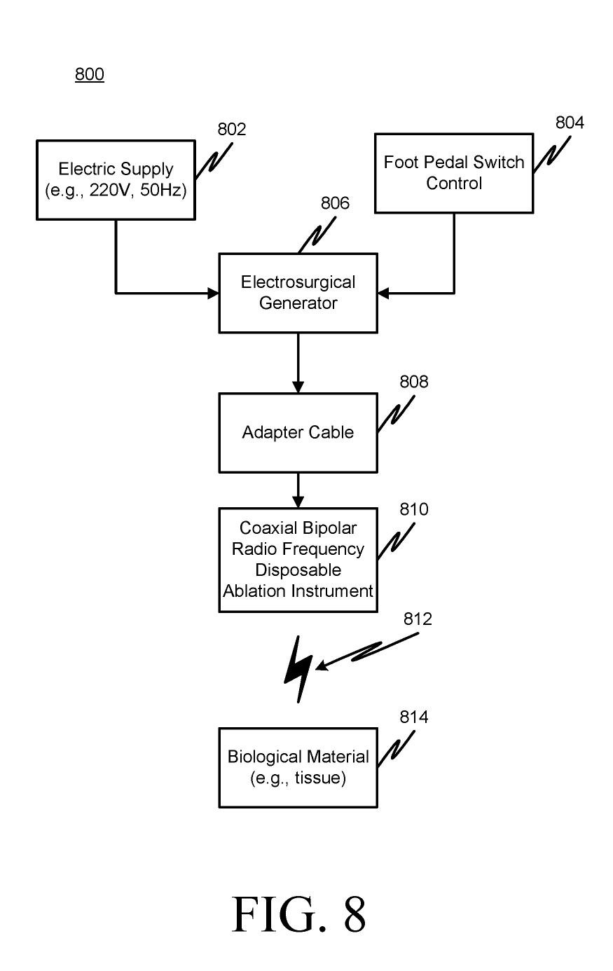

FIG. 8 is a diagrammatic illustration of an example ablating system 800 utilizing a coaxial electrocautery instrument 810 in accordance with some implementations. In particular, the system 800 includes an electric power supply 802 (e.g., a 220V, 50 Hz supply), foot pedal switch control 804, an electrosurgical generator 806, an adapter cable 808, a disposable coaxial bipolar RF electrocautery (ablation) instrument 810, which can produce radio frequency energy 812 that impinges on biological material 814 (e.g., tissue).

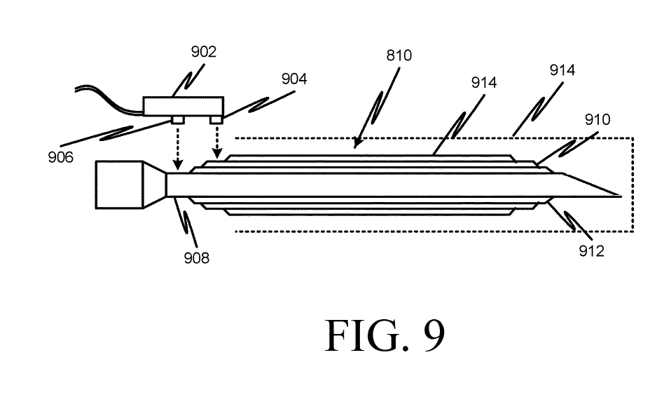

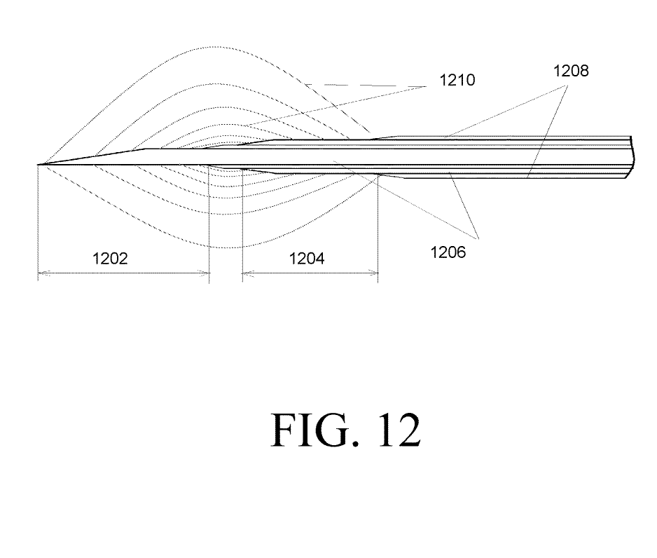

FIG. 9 is a diagram of an example disposable coaxial bipolar RF ablation instrument 810 (or needle). The instrument 810 can include an inner needle 908 that forms a first electrode, an outer needle 910 that forms a second electrode, an inner insulating layer 912 (between the inner and outer needles) and an outer insulating layer 914 (over the non-active portion of the outer needle 910). The instrument has two exposed contact areas: 1) a first exposed contact area near a proximate end of the instrument where a connector 902 connects to the instrument 810 and to an adapter cable 808 connected to an electrosurgical generator 806. The connector includes an outer needle contact 904 and an inner needle contact 906; and 2) a second exposed contact area on a distal end that forms the active portion of the instrument where RF energy is emitted into tissue. Details of the exposed active portion are shown in FIG. 12. The instrument 810 can also include a sleeve 914 to maintain the sterility of an insertion portion of the needle while the connector 902 is being attached. The sleeve 914 can be removed just prior to a surgical procedure.

There are several methods for creating an outer electrode on the inner needle. For example: 1. application of composition used in prosthetic dentistry (e.g., cermet), followed by baking it at high temperatures; 2. an electroplating method of applying a conductive layer on the previously applied insulating coating of the inner needle; 3. deposition of dielectric and conductive layers of relevant material ions in a vacuum environment using special deposition equipment; and 4. use of a second larger diameter needle in which the basic initial needle is inserted in through the appropriate dielectric layer. The fourth method is the currently preferred method.

The inner needles and outer needles can be stainless steel surgical needles of the appropriate gauges to permit the inner needle to fit inside the outer needle and sized for a given ablation procedure. The insulating material can be a medically safe material such as Mastersilver by a German company Renfert, which is a lacquer used in prosthetic dentistry, or other suitable insulating material.

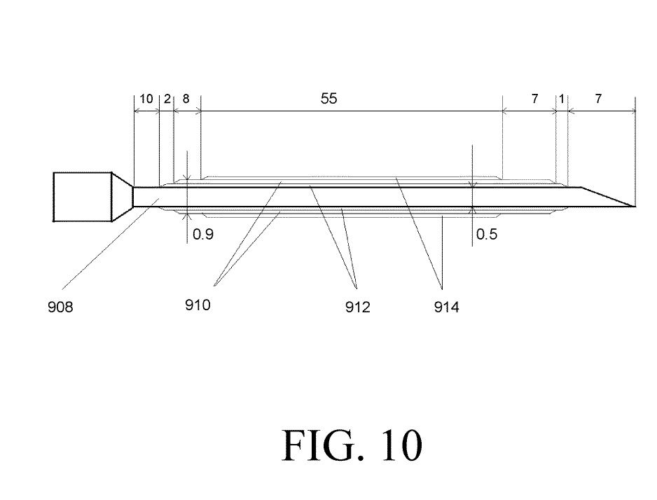

FIG. 10 is a diagram of an example coaxial electrocautery instrument (sized for thyroid tumor ablation) showing example dimensions in accordance with some implementations. The inner needle 908 can have a diameter of about 0.5 mm, the overall diameter of the outer needle Some implementations can include a coaxial bipolar ablation instrument comprising an outer needle and an inner needle inserted into the outer needle. The instrument can also include a first insulating layer disposed between the inner needle and the outer needle, and a second insulating layer disposed over a portion of the outer needle. The instrument can further include a first exposed region of the instrument disposed near a proximate end of the instrument, the first exposed region including a first exposed portion of the inner needle and a first exposed portion of the outer needle, the first exposed region constructed to provide contact to a connector, and a second exposed region of the instrument disposed near a distal end of the instrument, the second exposed region forming the active region of the instrument and including a second exposed portion of the inner needle and a second exposed portion of the outer needle.

Some implementations can include a coaxial bipolar ablation instrument comprising an outer needle and an inner needle inserted into the outer needle. The instrument can also include a first insulating layer disposed between the inner needle and the outer needle, and a second insulating layer disposed over a portion of the outer needle. The instrument can further include a first exposed region of the instrument disposed near a proximate end of the instrument, the first exposed region including a first exposed portion of the inner needle and a first exposed portion of the outer needle, the first exposed region constructed to provide contact to a connector, and a second exposed region of the instrument disposed near a distal end of the instrument, the second exposed region forming the active region of the instrument and including a second exposed portion of the inner needle and a second exposed portion of the outer needle.

can be about 0.9 mm. The overall length of the needle portion of the instrument can be about 90 mm. The first exposed portion (e.g., for the connector) can include a first exposed contact area of 10 mm, a 2 mm insulating spacer and an 8 mm contact area for the outer needle. The insulated main body of the instrument can be about 55 mm long. The second exposed portion (e.g., the active ablation area) can include an exposed outer needle portion of 7 mm, a 1 mm insulating spacer, and a 7 mm exposed end portion.

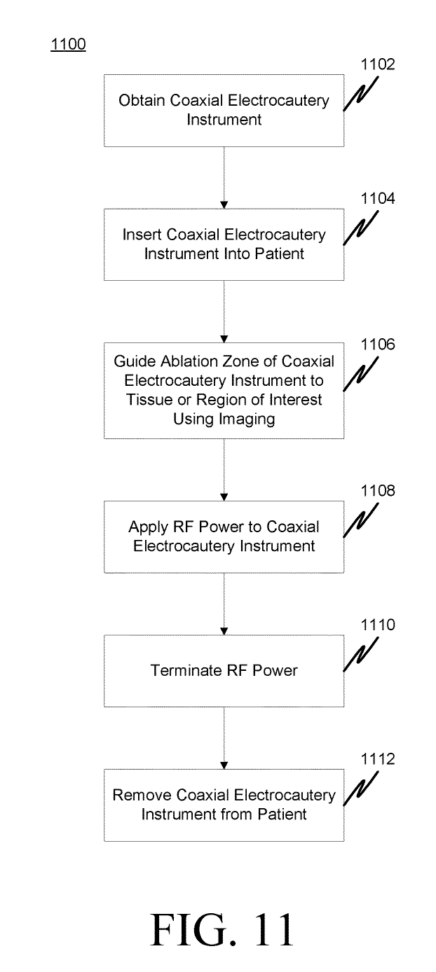

FIG. 11 is a flow diagram illustrating an example method 1100 of operation of the electrocautery instrument of FIG. 9 in accordance with some implementations. The process begins at 1102, where a disposable coaxial bipolar electrocautery instrument (e.g., 810) is obtained. If the instrument is not already connected to an electrosurgical generator, then attaching the instrument to an electrosurgical generator via an adapter cable. The process continues to 1104.

At 1104, the coaxial bipolar instrument is inserted into a patient. The process continues to 1106.

At 1106, the insertion of the coaxial bipolar instrument is guided to the region of interest by an imaging technique such as ultrasound or the like. The process continues to 1108.

At 1108, RF power is applied to the instrument. At 1110, the application of RF power is stopped or terminated. The termination of RF power can occur after a predetermined amount of time or after an amount of time based on the condition of the tissue. The process continues to 1112, where the coaxial bipolar instrument is removed from the patient. The instrument can optionally be discarded.

FIG. 12 shows that when applying the HF signal to the electrodes (1206, 1208) between their active parts (1202, 1204, respectively) a field is formed whose lines of force are indicated in blue (at 1210). At the same time the field force lines have the highest concentration at the site of convergence of active electrodes, the “antinode.” The resistance in this part of the tissue will be the smallest, since here the distance between the active portions of the electrodes is shortest. Accordingly, in the initial period of exposure the bulk of the HF generator input power will be allocated in this area. During the impact of high-frequency currents this area of the tissue will be breaking down and resistance to HF currents will increase. Since the resistance of this area increases the HF current with a higher concentration will flow through the surrounding areas, skirting the area of the maximum impact. Thus, increasing the time of action of HF signal, the maximum exposure area can increase in volume in the form of an ellipsoid, increasing in size along the tool axis.

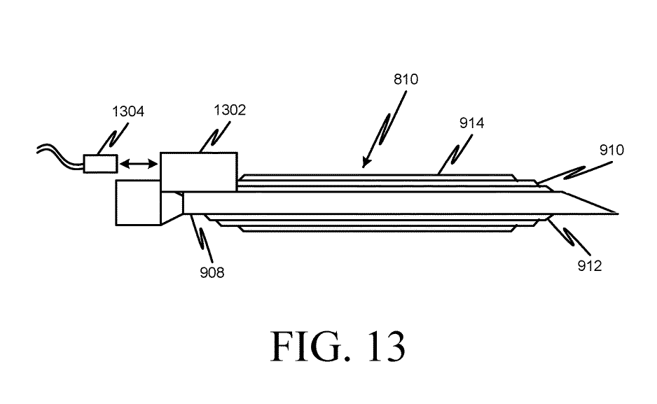

FIG. 13 shows an implementation in which the connector 1302 is integrated with the instrument 810 and a connector 1304 can be coupled to the connector 1302 on the instrument 810. This could increase the cost of the instrument 810, but may have advantages such as having an instrument manufactured with the connector 1302 attached at the factory.

It is to be understood that the description herein is exemplary only and is intended to provide an overview for the understanding of the nature and character of the disclosed subject matter. The accompanying drawings are included to provide a further understanding of various features and embodiments of the method and apparatus, which, together with their description serve to explain the principles and operation of the disclosed subject matter. Thus, while the disclosed subject matter has been described with reference to particular embodiments, it will be understood that the disclosed subject matter is not limited to the particular constructions and methods herein described and/or shown in the drawings, but also comprises any modifications or equivalents within the scope of the disclosed subject matter.

It is, therefore, apparent that there is provided, in accordance with the various embodiments disclosed herein, a radio frequency ablation instrument, system and method.

While the disclosed subject matter has been described in conjunction with a number of embodiments, it is evident that many alternatives, modifications and variations would be or are apparent to those of ordinary skill in the applicable arts. Accordingly, Applicant intends to embrace all such alternatives, modifications, equivalents and variations that are within the spirit and scope of the disclosed subject matter.

What is claimed is:

1. A coaxial bipolar ablation instrument comprising:
an outer needle;
an inner needle inserted into the outer needle;
a first insulating layer disposed between the inner needle and the outer needle;
a second insulating layer disposed over a portion of the outer needle;
a first exposed region of the instrument disposed near a proximate end of the instrument, the first exposed region including a first exposed portion of the inner needle and a first exposed portion of the outer needle, the first exposed region constructed to provide contact to a connector; and
a second exposed region of the instrument disposed near a distal end of the instrument, the second exposed region forming the active region of the instrument and including a second exposed portion of the inner needle and a second exposed portion of the outer needle.

2. The coaxial bipolar ablation instrument of claim 1, further comprising a connector coupled to the instrument and having a first contact contacting the first exposed portion of the outer needle and a second contact contacting the first exposed portion of the inner needle.

3. The coaxial bipolar ablation instrument of claim 1, wherein the inner needle and the outer needle are formed from stainless steel surgical needles.

4. The coaxial bipolar ablation instrument of claim 1, wherein the first insulating layer and the second insulating layer are formed from medical lacquer.

5. The coaxial bipolar ablation instrument of claim 1, wherein the second exposed region of the inner needle is about 7 mm and the second exposed portion of the outer needle is about 7 mm.

6. The coaxial bipolar ablation instrument of claim 1, wherein the first exposed portion includes an exposed portion of the first insulating layer.

7. The coaxial bipolar ablation instrument of claim 1, wherein the second exposed portion includes an exposed portion of the first insulating layer.

8. The coaxial bipolar ablation instrument of claim 1, wherein a diameter of the inner needle is about 0.5 mm.

9. The coaxial bipolar ablation instrument of claim 1, wherein a diameter of the outer needle is about 0.9 mm.

10. A high frequency ablation system comprising:
an electrosurgical generator;
a coaxial bipolar ablation instrument of claim 1;
an adapter cable having a first end constructed to be coupled to the instrument and a second end constructed to connect to the electrosurgical generator;
a power supply coupled to the electrosurgical generator; and
a control switch coupled to the electrosurgical generator.

11. The system of claim 10, further comprising a connector coupled to the instrument and having a first contact contacting the first exposed portion of the outer needle and a second contact contacting the first exposed portion of the inner needle.

12. The system of claim 10, wherein the inner needle and the outer needle are formed from stainless steel surgical needles.

13. The system of claim 10, wherein the first insulating layer and the second insulating layer are formed from medical lacquer.

14. The system of claim 10, wherein the second exposed region of the inner needle is about 7 mm and the second exposed portion of the outer needle is about 7 mm.

15. The system of claim 10, wherein the first exposed portion includes an exposed portion of the first insulating layer.

16. The system of claim 10, wherein the second exposed portion includes an exposed portion of the first insulating layer.

17. The system of claim 10, wherein a diameter of the inner needle is about 0.5 mm.

18. The system of claim 10, wherein a diameter of the outer needle is about 0.9 mm.
